(19) United States
(12) Patent Application Publication
(10) Pub. No.: US 2007/0032746 A1
(43) Pub. Date: Feb. 8, 2007

(54) Guide wire with magnetically adjustable bent tip and method for using the same

(75) Inventor: Jonathan C. Sell, Eagan, MN (US)

Correspondence Address: HARNESS, DICKEY, & PIERCE, P.L.C, 7700 BONHOMME, STE 400, ST. LOUIS, MO, 63105, US

(73) Assignee: Stereotaxis, Inc., St. Louis, MO (US)

(21) Appl. No.: 11/328,734

(22) Filed: Jan. 10, 2006

Related U.S. Application Data
(60) Provisional application No. 60/642,583, filed on Jan. 10, 2005.

(51) Int. Cl. A61M 25/00; A61M 31/00

(52) U.S. Cl. 600/585; 604/95.05

(57) ABSTRACT

The guide wire invention relates to improvements in magnetically navigable medical guide wires for enabling, in addition to magnetic navigation, conventional navigation without the use of a magnetic field. The distal portion of the guide wire may be navigated by either manually applying an axial rotation to the guide wire or by applying a magnetic field to modify the curvature of the distal portion to access small branch vessels in a subject body. The distal portion of the guide wire can also be straightened or aligned with the longitudinal axis of the guide wire by applying a magnetic field that straightens the bent section in the direction of the longitudinal axis, which enables the guide wire to push through a lesion.

### CROSS-REFERENCE TO RELATED APPLICATIONS

This application claims the benefit of U.S. provisional patent application Ser. No. 60/642,583 filed Jan. 10, 2005, the entire disclosure of which is incorporated herein by reference.

### FIELD OF THE INVENTION

This invention relates to guide wires for navigation of medical devices through body lumens such as blood vessels, and in particular to magnetically navigable guide wires for use in the vasculature.

### BACKGROUND OF THE INVENTION

Navigation of a conventional guide wire involves rotating or applying a torque to the proximal end of the guide wire repeatedly to rotate the bent end of the distal tip while the wire is pushed. This action is repeated until, by trial and error, the tip enters the desired vessel branch. In navigating through the vasculature of the body, the distal end of the conventional guide wire often comprises one or more bends that improve navigation through the vessels necessary to reach the target area for the medical intervention. Such pre-shaped guide wires have a high level of success in simple vessel anatomy. At the same time, the pre-shaped bends can become a disadvantage when the tip must access small vessels in the vasculature system or passages in the coronary anatomy. Furthermore, after the pre-shaped guide wire has made several bends, the guide wire becomes increasingly difficult to control, requiring repeated attempts to enter a desired vessel branch or gain passage through an occlusion. This trial and error method can frustrate the physician and cause additional wall contact and potential anatomical trauma.

To address these and other difficulties, magnetically navigable guide wires have been developed which can be controlled with the application of an external magnetic field. The user can advance the magnetically navigable guide wire into vessels with little or no contact between the end of the wire and the vessel wall. When the distal end of the guide wire is adjacent a branch vessel of interest, the user operates a magnetic system to apply a magnetic field (typically with the aid of a computerized user interface) to deflect the wire tip to align with the branch vessel. The magnet system can be made sufficiently accurate to direct the distal end of the guide wire into the branch on the first effort, eliminating the trial and error of manually operated guide wires and thereby reducing or eliminating trauma to the vessel wall. The deflection of the guide wire tip is controlled by the external magnets in magnetic navigation, and in normal use, the physician does not apply torque to the guide wire except in difficult turns. However, while magnetically navigable guide wires can be used to negotiate tortuous paths in the vasculature of a subject, negotiating simple vessel anatomy still requires navigation control, radiographic dye, X-ray fluoroscopy imaging and user interaction with the navigation system.

### SUMMARY OF THE INVENTION

The present invention relates to improvements in the construction of magnetically navigable medical guide wires to enable conventional navigation through simple vessel anatomy without the need for magnetic fields, and magnetic navigation through smaller complex vessel branches using an externally applied magnetic field. Generally, a guide wire constructed in accordance with the principles of this invention comprises an elongate wire having a proximal end and a distal end. The distal end further comprises one or more bent sections and one or more magnetically responsive elements disposed on the one or more bent sections of the guide wire. The magnetically responsive elements are preferably encapsulated or sealed by a radio-opaque material and secured to the bent section or sections by welding or with an adhesive. The magnetically responsive element is preferably comprised of a permanent magnetic material, but may alternatively comprise a permeable magnetic material. The guide wire comprises a core wire, and may further comprise a coil wire wound around the core wire along at least a portion of its length. The bent sections of the distal end of the guide wire may be subjected to an applied magnetic field to deflect and align at least one bent section with the longitudinal axis of the wire, which effectively straightens the distal end to enable the guide wire to align itself and pass through a lesion within a vessel which might otherwise “catch” the tip of the bend. The distal end may likewise be magnetically reoriented to gain access to a small vessel branch, by either removing or decreasing a previously applied magnetic field or by orienting the applied field to increase the curvature of the distal tip. The functional flexibility added by the magnetically available torque can, in conjunction with twisting of the proximal end of the guide wire, assist the physician in negotiating both sharp turns and tortuous paths within a vessel.

### BRIEF DESCRIPTION OF THE DRAWINGS

FIG. 1 is a side elevation view of a first preferred embodiment of a guide wire constructed according to the principles of this invention;

FIG. 2 is a side elevation view of a first preferred embodiment of a guide wire with the bent section aligned with the longitudinal axis of the wire by the application of a magnetic field;

FIG. 3 is a side elevation view of a first preferred embodiment of a guide wire showing the increased bent tip curvature obtained by application of a magnetic field of specific orientation;

FIG. 4 is a side elevation view of a second preferred embodiment of a guide wire constructed according to the principles of this invention;

FIG. 5 is a side elevation view of the second preferred embodiment of a guide wire with the bent section aligned with the longitudinal axis of the wire by the application of a magnetic field;

FIG. 6 is a side elevation view of the second preferred embodiment of a guide wire showing the increased bent tip curvature obtained by application of a magnetic field of specific orientation;

FIG. 7 is a side elevation view of a third preferred embodiment of a guide wire constructed according to the principles of this invention; and

FIG. 8 is a side elevation view of the third preferred embodiment of a guide wire with the bent tip curvature increased by the application of a magnetic field of specific orientation to work through the occlusion of a branch vessel.

### DETAILED DESCRIPTION OF THE INVENTION

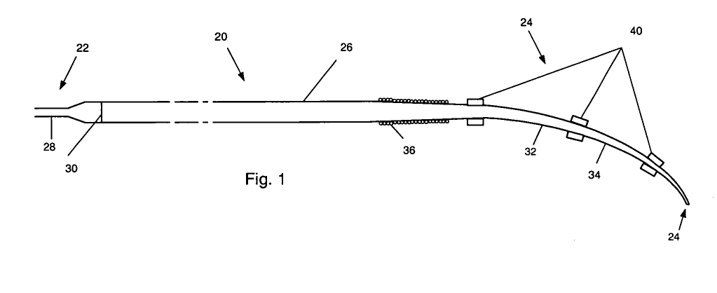

A first preferred embodiment of a magnetically navigable medical guide wire in accordance with the principles of this invention is indicated generally as 20 in FIG. 1. The guide wire 20 has a proximal end 22 and a distal end 24 and comprises a flexible core wire 26 extending from the proximal end substantially to the distal end. In the first preferred embodiment, the core wire 26 is between about 40 cm and about 350 cm, and tapers from a diameter of about 0.3 mm at the proximal end to about 0.05 mm at the distal end. In the preferred embodiment the bend 32 forms a bent distal section 34 that bends at an angle of between about 15 and about 90 degrees, and more preferably between about 20 and about 60 degrees.

The core wire 26 can be made of Nitinol, stainless steel or other suitable material, and may comprise a tapered cross-section that provides for increased flexibility near the tip of the guide wire. Additionally, the core wire can have a flat, malleable section that allows the tip of the guide wire to be shaped by the user.

The guide wire 20 may also comprise coil 36 around the core wire 26 along a portion of its length. The coil 36 can be made of a radio-opaque material useful for viewing in an X-ray or Fluoroscopic imaging system. Alternatively, or in addition, the guide wire 20 may also comprise a coating (preferably of a urethane or other polymer), which is loaded with radio-opaque material to enable viewing of the guide wire 20 in an X-ray or Fluoroscopic imaging system.

Referring to FIG. 1, disposed on the bent section 34 of the distal end 24 is at least one magnetically responsive element 40, of sufficient size, shape, and magnetization direction to align the bent section 34 relative with the direction of an applied magnetic field to access small branch vessels in the vasculature. The at least one magnetically responsive element 40 can be made of a permanent magnetic material or a permeable magnetic material, for enabling the distal end portion of the guide wire 20 to align in a selected direction when subjected to a magnetic field applied from an external source magnet. Suitable permanent magnetic materials include neodymium-iron-boron (Nd—Fe—B). Suitable permeable magnetic materials include Hiperco. The size and material of the magnetically responsive element 40 are selected so that the flexible distal end portion of the guide wire can be reoriented by the application of a magnetic field of no more than about 0.10 Tesla, and more preferably no more than about 0.08 Tesla, and still more preferably no more than about 0.06 Tesla.

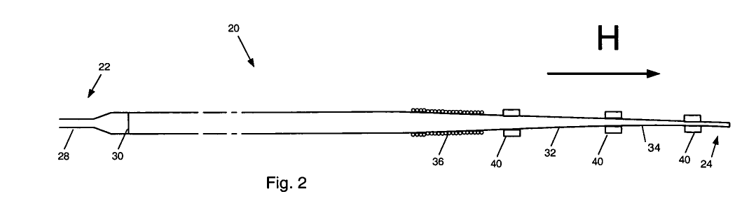

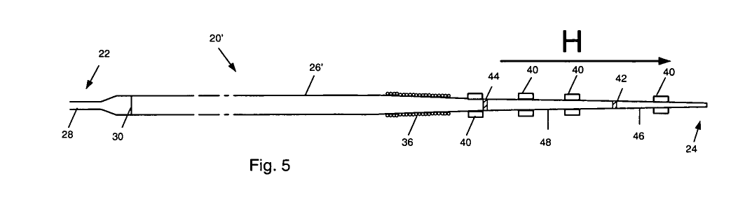

In the first preferred embodiment shown in FIG. 1, there are at least three magnetically responsive elements 40 on the guide wire, with two disposed on the bent section 34, and one disposed on the main section of the guide wire proximal to the bent section 34. The application of a magnetic field to the distal portion of the guide wire may act to straighten bend 34 section, as shown in FIG. 2, aligning the bent section 34 with the adjacent proximal section of the guide wire, or aligning the distal end portion in a selected direction as shown in FIG. 5.

In this first preferred embodiment, each magnetically responsive element 40 is preferably in the range of 1 to 2.5 millimeters long, and can be secured to the core wire 26 by laser welding, soldering, with an adhesive, or by any other suitable means of attachment. The magnetically responsive element 40 may have a slot, hole or groove through which the core wire 26 may be inserted to secure the element in place. It should be noted that an existing conventional pre-bent guide wire may be modified to include a magnetically responsive element secured to the pre-bent distal end section in accordance with the principles of the present invention. The guide wire 20 may also include a lubricious coating along its outside surface to allow for smooth tracking along vessel walls.

The guide wire 20 is sufficiently stiff that it can be advanced in the selected direction by pushing the proximal end of the guide wire 20, yet flexible enough that the guide wire can be deflected by an applied magnetic field to gain entry to a vessel branch. One way of determining guide wire deflection is by bending a fixed length, e.g. 0.5 inch. In the case of a magnetically navigable catheter, by holding the wire at a set distance proximal to the tip such as at 0.5 inch, and applying a magnetic field of known magnitude, H, at varying angles to the tip until the maximum tip deflection is observed. For example, in the Stereotaxis Niobe™ magnetic navigation system, a field of 0.08 Tesla can be applied within the subject in any direction. The maximum deflection angle of the guide wire in a 0.08 Tesla field is thus one way to characterize the guide wire performance in the Niobe™ magnetic navigation system. The inventors have determined that a minimum tip deflection angle of about 30 degrees from the pre-bent angle is desired for navigation of the guide wire according to the principles of the present invention.

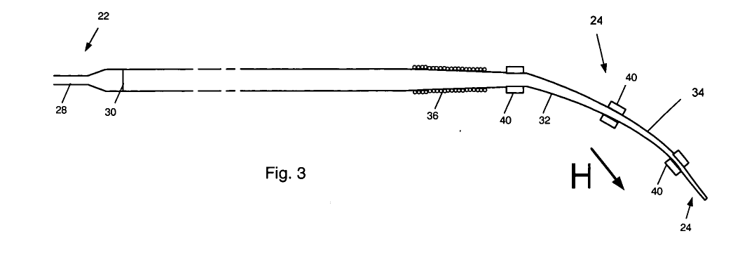

By applying a magnetic field in the appropriate direction, as shown in FIG. 2, the bent section 34 can be straightened or aligned with the longitudinal axis for enabling passage through a lesion in the vessel. The magnetic field can also be applied in a direction further away from the guide wire main axis to increase the curvature at the bent tip, as shown in FIG. 3 for the first preferred invention embodiment. The local magnetic field applies a torque to the guide wire tip which acts to direct the distal end in the direction chosen by the user, therefore facilitating navigation of the guide wire through tortuous or complex vessel anatomy.

The guide wire of the first preferred embodiment thus can be used in a bent orientation for conventional navigation without a magnetic field, yet can be straightened by an applied magnetic field to push through lesions within a vessel, or deflected by a magnetic field to access small vessel branches in the vasculature. The applied magnetic field that aligns the distal tip in a straightened orientation also holds the tip in the same orientation to provide support to the distal tip when pushing through a lesion, and improve the resistance to buckling.

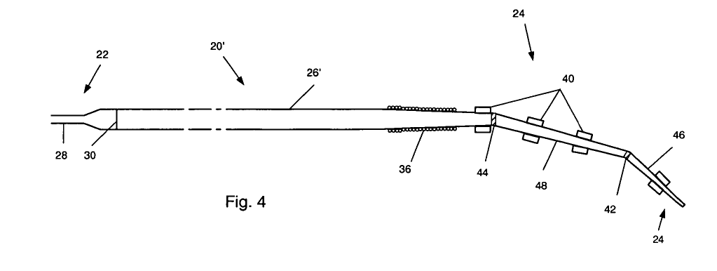

A second preferred embodiment of a magnetically navigable medical guide wire in accordance with the principles of this invention is indicated generally as 20′ in FIG. 4. The guide wire 20′ is similar in construction to guide wire 20, and corresponding parts are identified with corresponding reference numerals. The guide wire 20′ has a proximal end 22 and a distal end 24 and comprises a flexible core wire 26′ extending from the proximal end substantially to the distal end. In the second preferred embodiment, the core wire 26 is between about 40 cm and about 350 cm, and tapers from a diameter of about 0.3 mm at the proximal end to about 0.05 mm at the distal end.

In the second embodiment shown in FIG. 4, the distal end 24 of the core wire 26′ comprises a first bend 42 formed therein approximately 3 to 5 millimeters from the distal tip. In this embodiment, the bend 42 is at an angle of between about 15 and about 60 degrees, and more preferably between about 30 and about 35 degrees. The distal end 24 preferably also has a second bend 44 proximal of the first bend 42. In this embodiment the second bend 44 is at an angle of between about 15 and about 60 degrees, and more preferably between about 55 and about 65 degrees so that preferably the total of the two angles is between about 70 and 90 degrees. The first bend 42 defines a first bend section 46 between the bend 42 and the distal tip, and the first and second bends 42 and 44 define a bent section 48 between them.

The core wire 26′ can be made of Nitinol, stainless steel or other suitable material, and may comprise a tapered cross-section that provides for increased flexibility near the tip of the guide wire. Additionally, the core wire can have a flat, malleable section that allows the tip of the guide wire to be shaped by the user.

The guide wire 20′ may also comprise coil 36 around the core wire 26′ along a portion of its length. The coil 36 can be made of a radio-opaque material useful for viewing in an X-ray or Fluoroscopic imaging system. Alternatively, or in addition, the guide wire 20′ may also comprise a coating (preferably of a urethane or other polymer), which is loaded with radio-opaque material to enable viewing of the guide wire 20′ in an X-ray or Fluoroscopic imaging system.

Referring to FIG. 4, disposed on the bent section 46 of the distal end 24 is at least one magnetically responsive element 40, of sufficient size, shape, and magnetization direction to align the bent section 46 relative to the direction of an applied magnetic field to access small branch vessels in the vasculature. The at least one magnetically responsive element 40 can be made of a permanent magnetic material or a permeable magnetic material, for enabling the distal end portion of the guide wire 20′ to align in a selected direction when subjected to a magnetic field applied from an external source magnet. Suitable permanent magnetic materials include neodymium-iron-boron (Nd—Fe—B). Suitable permeable magnetic materials include Hiperco. The size and material of the magnetically responsive element 40 are selected so that the flexible distal end portion of the guide wire can be reoriented by the application of a magnetic field of no more than about 0.10 Tesla, and more preferably no more than about 0.08 Tesla, and still more preferably no more than about 0.06 Tesla.

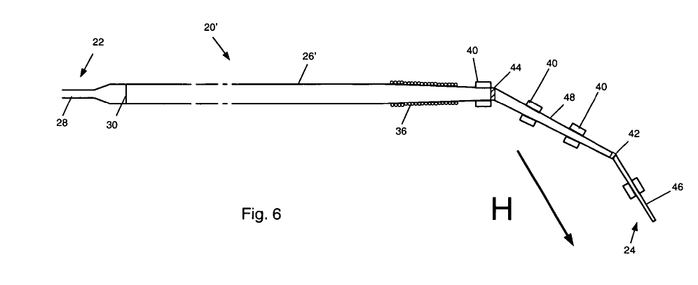

In the second preferred embodiment there are at least three, and as shown in FIG. 4, there are at least four magnetically responsive elements 40 on the guide wire, with two disposed on the bent section 46, two disposed on the bent section 48, and one disposed on the main section of the guide wire proximal to the bent sections 46 and 48. The application of a magnetic field to the distal portion of the guide wire may act to straighten bent sections 46 and 48, as shown in FIG. 5, aligning the bent sections 46 and 48 with the adjacent proximal section of the guide wire, or aligning the distal end portion in a selected direction as shown in FIG. 6.

In this second preferred embodiment, each magnetically responsive element 40 is preferably in the range of 1 to 2.5 millimeters long, and can be secured to the core wire 26′ by laser welding, soldering, with an adhesive, or by any other suitable means of attachment. The magnetically responsive element 40 may have a slot, hole or groove through which the core wire 26′ may be inserted to secure the element in place. It should be noted that an existing conventional pre-bent guide wire may be modified to include a magnetically responsive element secured to the pre-bent distal end section in accordance with the principles of the present invention. The guide wire 20 may also include a lubricious coating along its outside surface to allow for smooth tracking along vessel walls.

The guide wire 20′ is sufficiently stiff that it can be advanced in the selected direction by pushing the proximal end of the guide wire 20, yet flexible enough that the guide wire can be deflected by an applied magnetic field to gain entry to a vessel branch. One way of determining guide wire deflection is by bending a fixed length, e.g. 0.5 inch. In the case of a magnetically navigable catheter, by holding the wire at a set distance proximal to the tip such as at 0.5 inch, and applying a magnetic field of known magnitude, H, at varying angles to the tip until the maximum tip deflection is observed. For example, in the Stereotaxis Niobe™ magnetic navigation system, a field of 0.08 Tesla can be applied within the subject in any direction. The maximum deflection angle of the guide wire in a 0.08 Tesla field is thus one way to characterize the guide wire performance in the Niobe™ magnetic navigation system. The inventors have determined that a minimum tip deflection angle of about 30 degrees from the pre-bent angle is desired for navigation of the guide wire according to the principles of the present invention.

By applying a magnetic field in the appropriate direction, as shown in FIG. 5, the bent sections 46 and 48 can be straightened or aligned with the longitudinal axis for enabling passage through a lesion in the vessel. The magnetic field can also be applied in a direction further away from the guide wire main axis to increase the curvature at the bent tip, as shown in FIG. 6. The local magnetic field applies a torque to the guide wire tip which acts to direct the distal end in the direction chosen by the user, therefore facilitating navigation of the guide wire through tortuous or complex vessel anatomy.

The guide wire of the second preferred embodiment thus can be used in a bent orientation for conventional navigation without a magnetic field, yet can be straightened by an applied magnetic field to push through lesions within a vessel, or deflected by a magnetic field to access small vessel branches in the vasculature. The applied magnetic field that aligns the distal tip in a straightened orientation also holds the tip in the same orientation to provide support to the distal tip when pushing through a lesion, and improve the resistance to buckling.

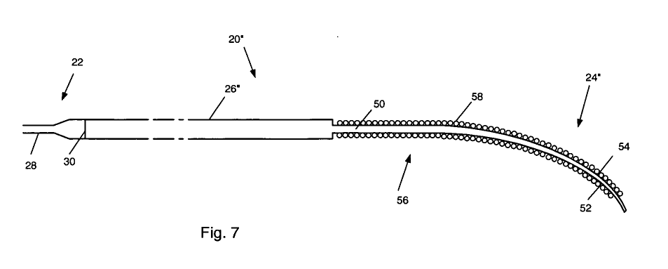

A guide wire constructed in accordance with a third preferred embodiment is indicated generally as 20″ in FIG. 7 and is generally similar in construction to guide wire 20, and corresponding parts are identified with corresponding reference numerals. The guide wire 20″ comprises a flexible core wire 26″ having a proximal end 22 and a distal end 24″ is shown in FIG. 7. The proximal end 22 of the guide wire 20 can include a shaft section 28 having a proximal landing 30, to which a core wire 26″ is attached. The distal end 24″ of the core wire 26″ comprises a flat wire section 50 having a bend 52 at approximately 3 to 5 millimeters from the tip and angled between 15 and 60 degrees, forming a bent distal tip section 54. The core wire 26″ can be made of Nitinol, stainless steel, or other suitable material or combination of materials. Surrounding the flat section 50 of the core wire 26″ is a magnetically responsive element 56 that is preferably a coiled wire 58 or sleeve made of a magnetically responsive material. The magnetically responsive material could be a permanent magnetic material or a permeable magnetic material, but in the preferred embodiment is a coiled permanently magnetized wire. The magnetically responsive coil 58 is coiled around the flat section 50 and the bent tip section 54 of the core wire 26″, preferably extending over both the bent section 54 and a portion of the straight portion of the guide wire proximal thereto. Alternatively, the magnetically responsive element 56 may comprise a sleeve made of a polymer manufactured with an angle set near the tip that encapsulates a permanently magnetized or magnetically permeable coiled wire. Suitable permeable magnetic materials include Hiperco. The size and material of the magnetically responsive element 56 are selected so that the bend 52 in the flexible flat section 50 of the core wire 26′ can be reoriented by the application of a magnetic field of no more than about 0.10 Tesla (and preferably no more than about 0.08 Tesla, and still more preferably no more than about 0.06 Tesla) to straighten or align with the longitudinal axis of the guide wire 20″. The guide wire of the second preferred embodiment thus can be used in a deflected or bent orientation for conventional navigation without a magnetic field, yet can be straightened by an applied magnetic field to push through lesions within a vessel.

The size, shape, and material of the magnetically responsive element 56 and the core wire 26″ are selected so that when a magnetic field of appropriate strength and direction is externally applied to the distal end of the guide wire 20″, the bent section 54 straightens relative to the proximal section of the guide wire, facilitating passage through straight sections of the vasculature, and in particular straight sections that have been narrowed by blockages. The size, shape, and material of the magnetically responsive element 56 and the core wire 26″ are selected so that when a magnetic field of appropriate strength and direction is externally applied to the distal end of the guide wire 20″ the distal end can orient in a selected direction to bypass obstructions in the vasculature and to make turns into selected branches of the vasculature.

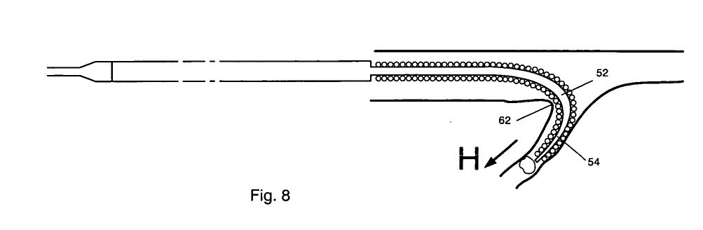

The guide wire of this second preferred embodiment thus can be used in a bent orientation for conventional navigation without a magnetic field, yet can be straightened by an applied magnetic field to push through lesions within a vessel, or deflected by a magnetic field to access small vessel branches in the vasculature. As shown in FIG. 8, the applied magnetic field that aligns the distal tip in a curved orientation also holds the tip in the same orientation to provide support to the distal tip when pushing through a lesion located past a vessel branch 62.

The above-described embodiments are intended to be illustrative only. For example, the conventional navigation technique of applying a torque to the proximal end of the guide wire may also be achieved by using a motor that is controlled by a physician. There are also numerous types of interventional magnetic procedures for which the guide wire described and the methods of controlling the guide wire are important. The invention can be readily adapted so that a physician, under guidance from an imaging system, uses the magnetic system to negotiate otherwise difficult turns and movements of the interventional device and to gain passage through a lesion. Application of a torque at the proximal end of the guide wire to effect a rotation of the distal tip can be used in combination with application of magnetic fields of various orientations and strength to increase the exploratory range of the guide wire tip. This aspect of the present invention can be used to improve navigation and to explore lesions to find the location most favorable for the guide wire progression. It will also be recognized that many of the inventive methods and apparatuses may be used in conjunction with any coil in a non-resonant circuit that applies a magnetic force on a suspended or embedded object that is magnetically moveable. Many other modifications falling within the spirit of the invention will be apparent to those skilled in the art. Therefore, the scope of the invention should be determined by reference to the claims below and the full range of equivalents in accordance with applicable law.

What is claimed is:

1. A elongate medical guide wire, comprising a core wire having a proximal end and a distal end, at least one bend adjacent the distal end forming at least one bent section, and a magnetically responsive element on at least one bent section of sufficient size and strength to change the angular relationship of the at least one bent section relative to the remainder of the guide wire upon the application of a magnetic field of no more than about 0.1 Tesla.

2. The guide wire according to claim 1 wherein there is one bend in the distal end of the guide wire forming one bend section, and there is at least one magnetically responsive element on the one bent section.

3. The guide wire according to claim 2 wherein the bent section is substantially straight.

4. The guide wire according to claim 2 wherein the bent section is curved.

5. The guide wire according to claim 2 wherein there are at least two magnetically responsive elements on the bent section.

6. The guide wire according to claim 5 wherein there is at least one magnetically responsive element on the core wire, proximal to the bend

7. The guide wire according to claim 1 wherein there is at least one magnetically responsive element on the core wire, proximal to the at least one bend.

8. The guide wire according to claim 1 wherein there are at least two bends, defining a first bent section between one bend and the distal tip, and a second bent section between the two bends.

9. The guide wire according to claim 1 wherein there is at least one magnetically responsive element on the first bent section.

10. The guide wire according to claim 1 wherein there is at least one magnetically responsive element on the second bent section.

11. The guide wire according to claim 1 wherein there is at least one magnetically responsive element on each of the first and second bent sections.

12. The guide wire according to claim 11 wherein there is at least one magnetically responsive element on the core wire, proximal to the bend

13. The guide wire according to claim 5 wherein there is at least one magnetically responsive element on the core wire, proximal to the bend

14. The guide wire according to claim 1 further comprising a coil of a radiopaque material disposed over the guide wire.

15. The guide wire according to claim 1 wherein the at least one magnetically responsive element is a permanent magnet.

16. The guide wire according to claim 1 wherein the at least one magnetically responsive element is a coil of magnetically responsive material disposed over the guide wire.

17. The guide wire according to claim 1 wherein the at least one bent section can substantially align with the proximal portion of the guide wire upon the application of a magnetic field of no more than about 0.1 Tesla in the appropriate direction.

18. The guide wire according to claim 1 wherein the angle between at least one bend section and the proximal portion of the guide wire can increase by at least 30° upon the application of a magnetic field of no more than about 0.1 Tesla in the appropriate direction.

19. The guide wire according to claim 1 further comprising a plastically deformable portion which can be bent to shape the distal portion of the guide wire.

20. A method of navigating a guidewire having a bend adjacent the distal end forming at least one bend section adjacent the distal end with at least one magnetically responsive element thereon, the method comprising applying a magnetic field to the at least one magnetically responsive element on the bent section to temporarily substantially align the bent section with the proximal portion of the guidewire to facilitate advancing the distal end of the guide wire.

21. A method of navigating a guidewire having a bend adjacent the distal end forming at least one bend section adjacent the distal end with at least one magnetically responsive element thereon, the method comprising applying a magnetic field to the at least one magnetically responsive element on the bent section to temporarily increase the angle of the bent section with the proximal portion of the guidewire to facilitate advancing the distal end of the guide wire in a new direction relative the axis of the guide wire.
